(19) United States
(12) Patent Application Publication
(10) Pub. No.: US 2017/0296301 A1
(43) Pub. Date: Oct. 19, 2017

(54) SYSTEM AND METHOD FOR MONITORING TAGGED ITEMS IN A MEDICAL FACITLITY

(71) Applicant: HALDOR ADVANCED TECHNOLOGIES LTD, Hod HaSharon (IL)

(72) Inventors: Guy DOR, Rosh Haayn (IL); Dan ZEEI, North York, Ontario (CA); Ilan KADOSH-TAMARI, Ramat Hasharon (IL)

(21) Appl. No.: 15/516,406

(22) PCT Filed: Sep. 7, 2015
(86) PCT No.: PCT/IL2015/050901

§ 371 Date: Apr. 2, 2017

(51) Int. Cl. A61B 90/98; A61B 90/00; A61B 34/20; G08B 21/24

(52) U.S. Cl. CPC A61B 90/98; A61B 34/20; A61B 90/37; A61B 2034/2051; A61B 2034/2072; G08B 21/24; A61B 2090/0805

(57) ABSTRACT

The subject matter discloses a system for monitoring tagged items in a medical facility, comprising one or more reader units configured to collect and store tracking data related to tagged items in real time, said monitoring data includes radio frequency tag identification data unique for each tagged item; a medical facility server configured to receive the monitoring data from the one or more reader units, determine whether one or more predetermined rules or conditions are met, and trigger an action based on said determination; wherein the monitoring data comprises identification data and utilization data related to tagged items which are intended for use in the medical facility.

### FIELD OF THE INVENTION

The subject matter relates generally to a system and method for monitoring tagged items in a medical facility.

### BACKGROUND

Systems for monitoring tagged items, such as sponges, instruments and towels, are critical in ensuring the tagged items are accounted for during medical procedures. Systems are set up to scan and track the tagged item by scanning a tag attached to the tagged item. The tag may be a radio frequency (“RF”) tag, etching, 2D marking, a barcode, or the like.

Some existing products enable identification of retained surgical sponges only, to detect the tagged sponge. Other products enable scanning a barcode or 2D marking of a tagged sponge and/or towel before and after use in the operation room. Each barcode and or 2D marking is unique, which enables identifying each tagged sponge and/or towel individually. The barcode and/or 2D marking enables identifying what type of tagged sponge and/or towel is tagged with the barcode and/or 2D marking. Such products enable counting a total quantity of the tagged sponges and/or towels that remain in the operating room after actual surgery is completed, and just prior to the formal announcement by the OR team that the specific medical procedure, e.g. surgery, is officially completed, by scanning each barcode and/or 2D marking of the tagged sponge and/or towel before and after the medical procedure. When the barcode and/or 2D marking of a tagged sponge and/or towel is scanned prior to the medical procedure, but is not scanned afterwards when all other barcodes and/or 2D markings are scanned, the person in the OR scanning the sponge and/or towel is alerted that a tagged sponge and/or towel is missing.

Other products provide both identification of retained surgical sponges, and counting and reconciliation of tagged sponges and towels before and after a medical procedure. Such products count tagged sponges and towels used during a medical procedure and may provide information of the type of tagged sponge and or towel attached to a tag. The tags connected to the tagged sponges and or towels are radio frequency identification (“RFID”) tags, which enable identification of tagged sponges by scanning the RFID tag via scanning devices, such as antennas, bucket antenna, handheld scanners or the like.

Current products perform: (i) counting & reconciliations of tagged sponges and towels, (ii) determination of Retained Surgical Items (“RSI”), but only for sponges and towels), or (iii) counting & reconciliations of tagged sponges and towels including determination of RSI only for sponges and towels.

### SUMMARY

It is an objective of the subject matter to disclose a system for monitoring tagged items in one or more operating rooms in a medical facility, comprising one or more reader units configured to collect and store tracking data related to tagged items in real time, said monitoring data includes radio frequency tag identification data unique for each tagged item; a medical facility server configured to receive the monitoring data from the one or more reader units, determine whether one or more predetermined rules or conditions are met, and trigger an action based on said determination; wherein the monitoring data comprises identification data and utilization data related to tagged items which are intended for use in the medical facility.

In some cases, each reader unit of the one or more reader units comprises a radio frequency identification (RFID) input unit configured to obtain monitoring data stored in a RFID tag connected to a tagged item; said monitoring data comprises a tag identification data for identifying the tagged item; a reader unit processor configured to continuously update a surgery tag list of tagged items in the operating room based on tag identification data; and a reader unit database configured to store the monitoring data and utilization data of the tagged item.

In some cases, the medical facility server comprising a central database for storing the set of rules and conditions; a central processor for analyzing collected monitoring data to determine whether an action should be triggered according to a predetermined protocol.

In some cases, the system further comprising one or more locator antennas configured to penetrate human tissue and scan tagged items located within human tissue, wherein said one or more locator antennas are operationally connected to a reader unit of the one or more reader units.

In some cases, the system further comprising one or more handheld scanners configured to scan tagged items in an operating room; said one or more handheld scanners are operationally connected to a reader unit of the one or more reader units.

In some cases, tagged items are scanned using a handheld scanner of the one or more handheld scanners to add a tagged item to the surgery tag list.

In some cases, tagged items are scanned by a handheld scanner of the one or more handheld scanners to remove a tagged item from the surgery tag list.

In some cases, the reader unit processor is configured to determine whether a tagged item is missing during a medical procedure or after completion thereof.

In some cases, the central processor is configured to determine whether a number of tagged items of a specific type which were used in a medical procedure is larger than a predetermined threshold.

In some cases, the system further comprising one or more mobile controllers configured to communicate with the one or more reader units and with the medical facility server, said communication enabling the mobile controller to receive notifications from the medical facility server and from the reader unit, said notifications indicating that an action is required.

In some cases, the central processor is configured to trigger generation of a notification to one or more mobile controllers, based on said determination.

In some cases, the mobile controller is configured to initiate a command to the reader unit for activating a locator antenna configured to penetrate human tissue and scan RFID tags located within human tissue when a tagged item is determined as missing during a medical procedure.

It is another object of the subject matter to disclose within a computerized system a method for monitoring tagged items in a medical facility, the method comprising obtaining monitoring data from one or more reader units located in one or more operating rooms, determining, based on the monitoring data, whether one or more predetermined rules or conditions are met, and generating alerts based on said determination; wherein the monitoring data comprises identification data and utilization data related to tagged items which are intended for use the medical facility.

In some cases, the method further comprising continuously updating the monitoring data during the medical procedure.

In some cases, the monitoring data comprises RFID tag identification data obtained by the one or more reader units by scanning the tagged item.

In some cases, the reader unit obtains the tag identification data from a handheld scanner.

In some cases, the reader unit obtains the tag identification data from a locator antenna configured to penetrate human tissue and scan RFID tags located within human tissue.

In some cases, the method further comprising automatically activating the locator antenna when a tagged item is determined as missing.

In some cases, the method further comprising associating tagged item information to tag identification data and storing the associated information.

### BRIEF DESCRIPTION OF THE DRAWINGS

Exemplary non-limited embodiments of the disclosed subject matter will be described, with reference to the following description of the embodiments, in conjunction with the figures. The figures are generally not shown to scale and any sizes are only meant to be exemplary and not necessarily limiting. Corresponding or like elements are optionally designated by the same numerals or letters.

FIG. 1 is a schematic illustration of a system for monitoring tagged items, according to some exemplary embodiments of the subject matter;

FIG. 2 is a schematic illustration of a reader unit for monitoring tagged items, according to some exemplary embodiments of the subject matter;

FIG. 3 is a schematic illustration of a medical facility server, according to some exemplary embodiments of the subject matter;

FIGS. 4A-4B are flow charts of methods for scanning of RFID tags of tagged items, according to some exemplary embodiments of the subject matter;

FIG. 5 is flow chart of a method for intermediate item counting, according to some exemplary embodiments of the subject matter;

FIG. 6 is flow chart of a method for identifying item types via a central database, according to some exemplary embodiments of the subject matter;

FIG. 7 is flow chart of a method for generating a notification for of a detected event, according to some exemplary embodiments of the subject matter

FIGS. 8A-8B are flowcharts of a method for synchronizing a medical facility server to other components, according to some exemplary embodiments of the subject matter;

FIG. 9 shows a flowchart of a method for providing a surgery check list by a mobile controller, according to some exemplary embodiments of the subject matter; and,

FIG. 10 shows a flowchart of a method for providing a surgery check list by a medical facility server, according to exemplary embodiments of the subject matter.

### DETAILED DESCRIPTION

The subject matter discloses a centralized system and method for monitoring tagged items across multiple locations of a medical facility, e.g. in one or more operating rooms, sterile processing rooms, storage rooms, etc., according to exemplary embodiments.

One technical problem dealt by the disclosed subject matter is providing a system that enables providing alerts and distributing information related to tagged items across multiple locations, e.g. a plurality of operating rooms, in one or more medical facilities to enable proper response in case of an emergency or a critical event. Those skilled in the art are aware that no current technology and/or product performs count and reconciliation for all surgical items, e.g. sponges, towels and surgical instruments, and or can identify RSI over all surgical items e.g. sponges, towels and surgical instruments, or perform both counting and reconciliations along with RSI for all surgical items, which is a mandatory counting and reconciliations procedure requirement by all global healthcare regulators. Furthermore, there is no product or technology that perform such activities for multiple locations simultaneously. Rather, most products are stand-alone and localized, and cannot communicate and or integrate with other operating rooms and/or with other systems at a hospital, or with external healthcare systems (i.e. for regulatory auditing and reporting purposes) nor can they be centrally monitored and or configured so that they can, for example meet one or more predetermined rules or conditions, and which are triggered by actions based on the counting, reconciliations, unitization, handling and RSI of such surgical items a growing need exists for providing healthcare systems that can enhance public health care by providing up-to-date, centralized data. Embodiments of the present disclosure allow central management, audit item or tool tracking and integration with other healthcare systems, thus providing optimal clinical safety, logistics, productivity, regulatory compliance and reporting.

One technical solution according to the disclosed subject matter is implementing a system in a medical facility, the system comprising a medical facility server configured to synchronize, monitor and communicate with one or more reader units located in operating rooms of the medical facility, in order to maintain continuously updated monitoring and utilization data which may be accessed via the medical facility server or a device which is operationally connected to the medical facility server. The reader units provide on-site monitoring and management of the number of tagged items, e.g. disposable sponges, towels, instruments etc., located in an operating room during a medical procedure. Each reader unit is connected to the medical facility server, which collects, synchronizes, and manages information received from the one or more reader units. The medical facility server synchronizes and updates the data received from the one or more reader units at predetermined times or according to a set of rules and conditions. The medical facility server stores the continuously updated monitoring and utilization data, then analyzes the data and provides alerts or notifications to medical staff when it is determined that the obtained data meets one or more predetermined conditions, or triggers a rule requiring attention of the medical staff.

Embodiments of the invention may include an article, such as a non-transitory computer or processor readable storage medium, e.g. a memory, a disk drive, or a USB flash memory encoding, including or storing instructions, e.g., computer-executable instructions, which when executed by a processor or controller, cause the processor or controller to carry out methods disclosed herein. The instructions may cause the processor or controller to execute processes that carry out methods disclosed herein. The processes and displays presented herein are not inherently related to any particular computer or other apparatus. Various general purpose systems may be used with programs in accordance with the teachings herein, or it may prove useful to construct a dedicated device to perform the desired method. It will be appreciated that a variety of programming languages may be used to implement the embodiments of the invention as described herein.

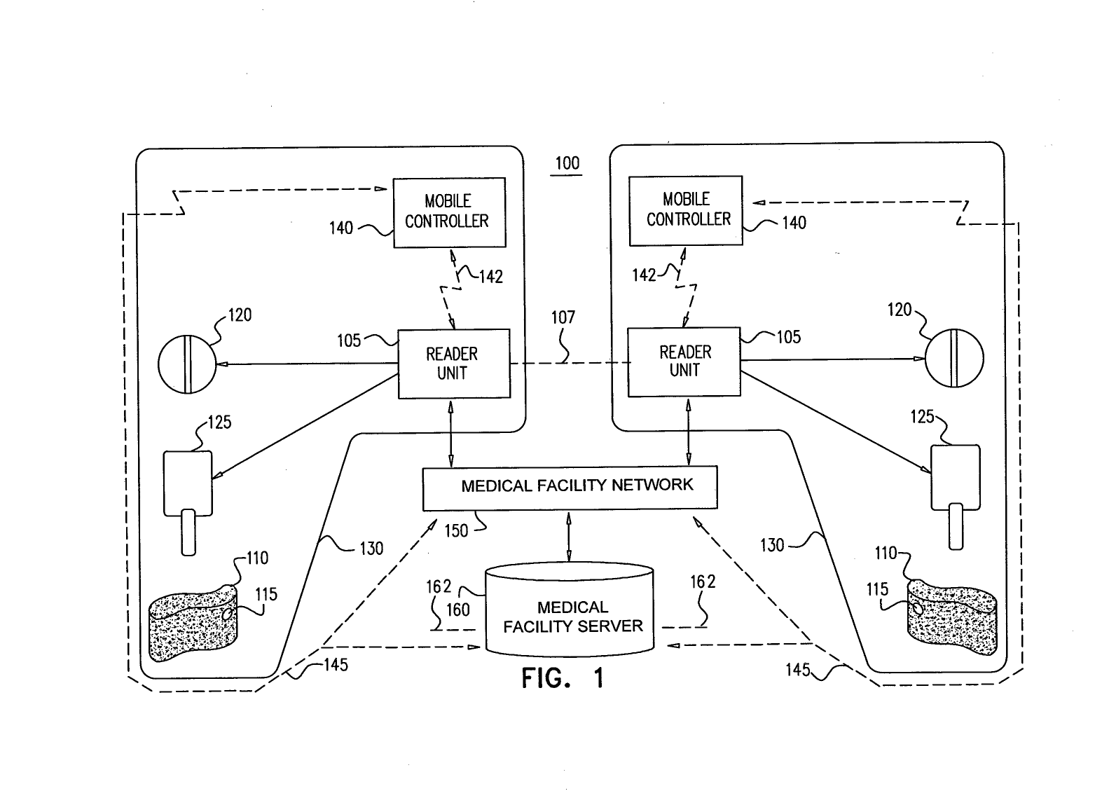

FIG. 1 shows a system for monitoring tagged items, according to some exemplary embodiments of the subject matter. The system 100 is implemented in a medical facility, which comprises one or more operating rooms, represented here as two instances of operating rooms 130. In some cases, the operating room 130 may be a perioperative department and/or a sterile processing department. The system 100 comprises one or more reader units 105, represented here by two instances of reader units 105, signifying any number of reader units, represented by the dotted line 107. At least one reader unit 105 of the one or more reader units 105 is located in each operating room 130.

The reader unit 105 is configured to perform on-site monitoring of one or more tagged items 110, represented by two instances of tagged items 110, in the operating room 130. Each tagged item 110 of the one or more tagged items 110 comprises an RFID tag 115. The RFID tags 115 may be attached to the one or more tagged items 110, for example during the manufacturing process of the items 110, and each RFID tag 115 comprises tag identification data that includes information related to the item 110 it is attached to. In some exemplary embodiments of the subject matter, the RFID tag 115 may alternatively be a tag comprising etching, 2d-marking, barcode, or the like. In some cases, the RFID tag 115 also comprises etching, 2d-marking, barcode, or the like.

The tag identification data includes data that enables identifying the tagged item 110, when the tagged item 110 is scanned with a scanner or antenna. The tag identification data may include a serial number, e.g. within a predetermined range of values, for example a production number within a specific range. For example, the production number of a certain batch of disposable sponges may range from 1,000,000 to 1,100,000, where each disposable sponge is provided with a unique production number that may be stored in the RFID tag 115 attached to a sponge, as the tag identification data for identifying and monitoring each disposable sponge.

In some non-limiting embodiments the tag identification data may comprise several different identification values, e.g. batch number, manufacturing ID, unique hospital identification number, FDA's UDI or the like. Each tag identification data uniquely identifies each tagged item 110.

The one or more reader units 110 obtains monitoring data of the tagged items 110 in the operating room 130. The monitoring data comprises identification data and tagged item information, to identify and track tagged items 110, e.g. using the tag identification data as well other identifiers or information related to a tagged item. The monitoring data may be stored in a storage unit, e.g. in reader box 105 and/or in a central storage unit that may be operationally connected to a central hospital server. The monitoring data may be used to generate a surgery tag list as described herein.

Tagged item information may comprise general data related to the tagged item, e.g. a type of tagged item (e.g. a sponge, a towel, a surgical tool, etc.), the type of medical procedure for which the tagged item 110 is designated or used, manufacturing date of the tagged item 110, expiration date of the tagged item 110, composition of the tagged item 110, the manufacturer, and the like. The tagged item information may further comprise utilization, reprocessing instructions and status, storage and/or usage data, e.g. in which operating room 130 the tagged item 110 is intended to be used, which medical case is associated with the tagged item 110, the medical team who is participating in the medical procedure, physician item request, scanning history of the tagged item 110, time stamps of the tagged item 110 being scanned, etc.

Scanning history includes data obtained during a scanning of the tagged item 110, e.g. scanning tagged items when they are received at the medical facility and transferred into storage or to certain medical units, when a tagged item 110 is inserted or removed from a surgery tag list, scanning of a tagged item 110 during an intermediate scanning performed during a medical procedure, scanning by a locator antenna 120 if a tagged item 110 is determined to be missing, etc. The scanning history may include date and time of the scan, the location where the scan was performed, the person who performed the scan, the device that was used during the scan, etc.

The reader unit 105 communicates with one or more mobile controllers 140, represented here as two instances of mobile controllers 140, signifying any number of mobile controllers 140. A mobile controller 140 is configured to enable the medical staff, e.g. a nurse, to control and manage the reader unit 105. The mobile controller 140 may be a tablet, a personal computer, a smart phone, etc. The mobile controller 140 communicates with the reader unit 105 through a wireless network 142, for example direct WiFi or via the hospital's WiFi network. It is appreciated by one skilled in the art that the mobile controller 140 may communicate with any reader unit 105 of the one or more reader units 105, and one mobile controller 140 may communicate with multiple reader units 105.

The reader unit 105 is operationally connected to one or more locator antennas 120, represented here by two instances of locator antennas 120. The one or more locator antennas 120 are configured to scan through human tissue in order to detect if a tagged item 110 was accidentally left in a patient during a medical procedure performed in the operating room 130. The reader unit 105 is connected to a predetermined number of locator antennas 120 to enable optimal coverage of the operating room 130, e.g. the reader unit 105 may be connected to three locator antennas 120. Each locator antenna 120 maybe independently activated by the reader unit 105 according to the coverage area necessary for locating a missing tagged item. In some non-limiting embodiments, the locator antenna 120 is mobile and designed to travel through the operating room to access all areas.

The reader unit 105 is connected to one or more handheld scanners 125, represented by two instances of handheld scanners 125. A handheld scanner of the one or more handheld scanners 125 is configured to scan the tagged item 110 to obtain the tag identification data from the RFID tag 115. The handheld scanner 125 may be operated by the medical staff, e.g. by a nurse, who scans the tagged item 110 that is to be entered into or removed from the operating room 130. For example, the nurse scans each tagged item 105 as it is placed on a cart to be wheeled into the operating room 130, before initiation of the medical procedure.

Each tagged item 110 that is scanned by a handheld scanner 125 may be stored, for example inserted into a surgery tag list, which enables monitoring the tagged items 110 and ensuring each tagged item 110 is accounted for during the medical procedure and after it is completed. The tagged item 110 may be scanned again in order to remove the tagged item 110 from the tag surgery list, upon or after the tagged item 110 is removed from the operating room 130 (e.g., during the medical procedure or after it).

A total count of tagged items 110 that were used in a certain medical procedure, or that are currently in use during a medical procedure, may be obtained using the surgery tag list. The total count is a calculated sum of the total amount of tagged items 110 inserted into the surgery tag list. The total count is updated each time tagged items 110 are added or removed from the surgery tag list. Continuously updating the surgery tag list enables monitoring the tagged items 110 in the operating room 130. A total count of the tagged items 110 scanned for entering the operating room prior to commencement of the medical procedure may be provided, e.g. displayed on a display unit of a mobile controller 140. Similarly, a total count of the tagged items 110 scanned for removal from the surgery tag list during or after the medical procedure may be provided. The total count of entered tagged items may be compared by the reader unit 105 to the total count of removed tagged items, and if it is determined that an item is missing, a notification may be automatically provided to the medical staff, in real time.

The surgery tag list may be or may include one or more data structures, e.g. a table, chart, database, or any other data entity, that stores data related to a certain medical procedure performed in a certain operating room. The surgery tag list may include tag identification data such as an item's serial number or ID, FDA's UDI and information related to the tagged item such as the item type, manufacturer, batch number, expiration date, etc. The surgery tag list may further include data related to the procedure, such as the time and date of the planned procedure, the attending physician or surgeon, participating staff members, assigned operating room, etc. In some exemplary embodiments of the subject matter, the surgery tag list comprises items that are not tagged with any marking or serializing technology, which require manual entering of the item into the surgery tag list as well as manually removing the item prior to closure of the medical procedure. In some non-limiting embodiments, the surgery tag list may also include exceptions reported by the clinical staff related to any special event that requires further attention by other staff members, for history maintenance or reporting purposes as well.

During a medical procedure, a scrub nurse, who is present in the operating room 130 during the medical procedure, may perform an intermediate count of the tagged items 110 present in the operating room. The intermediate count is performed using the handheld scanner 125 located in the operating room, which may be covered with a sterile bag to prevent contamination of the tagged item 110 by the handheld scanner 125. The scrub nurse operating the handheld scanner 125 may scan the tagged items 110 in the operating room 130, in order to continuously update the surgery tag list that maintains all items that are in use during the medical procedure, and notify in case of a discrepancy, thus to ensure no tagged item 110 remains inside a patient's body during or after the medical procedure. When the intermediate scan is performed, the tagged items 110 are scanned and the tag identification data associated with each tagged item 110 is compared to the tag identification data listed in the surgery tag list. After completion of the intermediate scan of the tagged items 110, the reader unit 105 determines whether an intermediate total count of tagged items 110 scanned equals to the total count of the surgery tag list while comparing the detected tag identification data with the tag identification data listed in surgery tag list. If the total counts are not equal, the reader unit 105 provides a notification of a missing tagged item. The notification may be distributed to one or more mobile controllers 140, to inform the medical staff of a necessary action, for example activating the locator antennas 120 to locate missing tagged items.

The scrub nurse or other medical personnel may activate the handheld scanner 125 to remove the tagged items 110 from the surgery tag list, as the tagged items 110 are removed from the operating room 130 (e.g. during or after completion of the medical procedure). When a tagged item 110 is determined as missing or unaccounted for after scanning the tagged items 110 using the handheld scanner 125, activation of the locator antennas 120 may enable locating the missing tagged item 110 and determining whether it remained inside the patient.

The mobile controller 140 controls and manages the reader unit 105. For example, the mobile controller 140 transmits a command to the reader unit 105 to obtain monitoring data from the one or more locator antennas 120. Since mobile controller 140 may be connected to any one of the reader units from the one or more reader units 105 through a wireless network 142, the medical staff using the mobile controller 140 is able to connect to each reader unit 105. For example, one nurse may be required to review the count of tagged items in two different operating rooms 130, by connecting the mobile controller 140 to the reader unit 105 of each of the operating rooms 130.

The one or more reader units 105 are connected to one or more medical facility servers 160, represented here by a medical facility server 160, signifying any number of medical facility servers, via a hospital network 150, e.g. a local area network, wide area network, Ethernet, etc. In some non-limiting embodiments of the subject matter at least one of the one or more hospital servers 160 is configured on a cloud. The medical facility server 160 is configured to analyze the surgery tag lists maintained and provided by the one or more reader unit 105. The medical facility server 160 stores the surgery tag lists and analyzes each list to determine required actions, for example activation of certain protocols to enable the medical staff to operate more efficiently. The medical facility server 160 stores a set of predetermined rules and/or conditions, which may be checked during the analysis of the surgery tag lists obtained from the one or more reader units 105. If one or more rules and/or conditions are met, certain actions, notifications or alerts may be automatically triggered by the central server 160.

Whenever the surgery tag list is updated by the reader unit 105, the reader unit 105 stores the changes and transmits the updated surgery tag list to the medical facility server 160. In some exemplary embodiments of the subject matter, the medical facility server 160 is connected to the one or more reader units 105 and the mobile controller 140 through the wired network, e.g. LAN or WAN, or via a wireless network 142.

When the medical facility server 160 receives an updated surgery tag list from the reader unit 105, it may analyze and determine whether one or more rules or conditions from a set of predetermined rules and conditions has been met. If a rule or condition has been met, the medical facility server 160 may trigger certain actions or procedures accordingly. For example, if the analysis of the surgery tag list indicates that an excessive number of tagged items 110 are used in a certain operating room 130, the medical facility server 160 may generate an alert or notification to the medical staff to assist in the medical procedure. The alert may be received by a nurse using a mobile controller 140, which may be located at a different location (e.g., a different operating room) in the medical facility. When the mobile controller 140 displays the alert received from the medical facility server 160, the nurse is informed that he/she is immediately required for assistance in the operating room 130. In another example, the notification may indicate that a specific doctor is required for assistance. In some non-limiting embodiments, the mobile controller 140 connects and communicates with the medical facility server 160 directly via network connection 145, which may include e.g. a wired network and/or a wireless network, cellular network, etc.

In some exemplary embodiments of the subject matter, the medical facility server 160 provides tagged item information, which provides additional information related to a tagged item 110, which may be associated with the tag identification data in the surgery tag list. The tagged item information is transmitted to the reader unit 105 to be associated with the tag identification data when the tagged item 110 is scanned. The tagged item information may comprise a type of the tagged item 110, and information related to the medical procedure in which the tagged item was used or is to be used, e.g. a medical case identification number, comments related to the medical case, personnel required to be present during the medical procedure, date and time of the medical procedure, location of the medical procedure, and the like. The tagged item information is stored with the associated tag identification data in the surgery tag list, and the updated surgery tag list is then transmitted and stored in the medical facility server 160. The medical facility server 160 may analyze the updated surgery tag list and may use the tagged item information to analyze and determine whether a rule or condition of the set of rules and conditions is met.

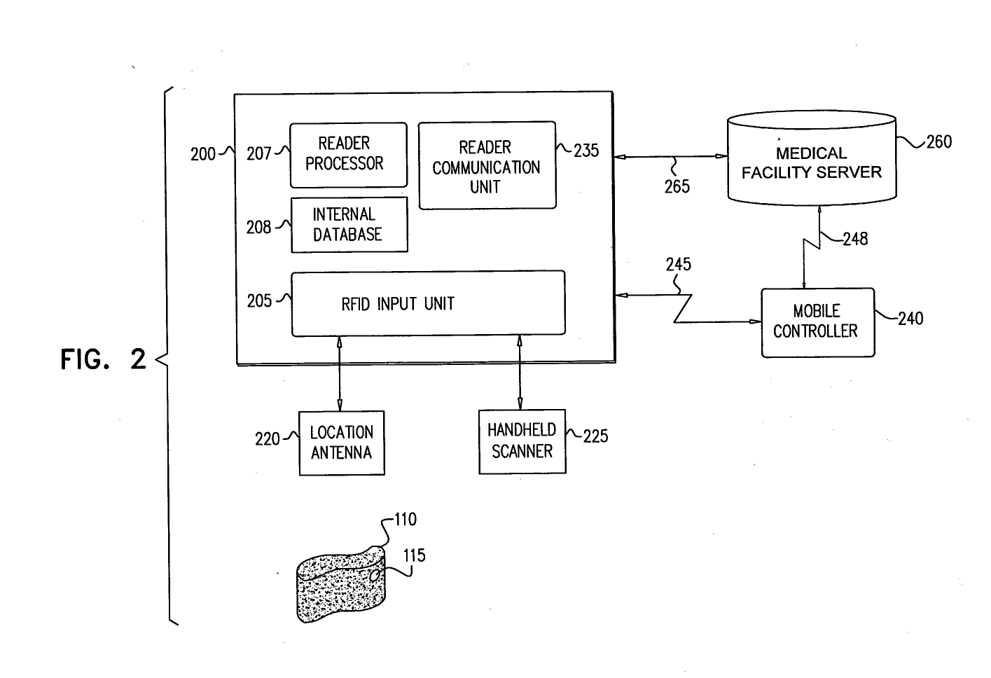

FIG. 2 shows a reader unit for monitoring tagged items in an operating room, according to some exemplary embodiments of the subject matter. The reader unit 200 comprises a radio frequency identification input unit (“RFID input unit”) 205, which receives scanned tag identification data from a locator antenna 220 or a handheld scanner 225.

The reader unit 200 comprises a computing processor 207, which is configured to manage the monitoring data obtained by the RFID input unit 205. The computing processor 207 is configured to update the surgery tag list by inserting tagged items 110 to surgery tag list, removing tagged items from the surgery tag list, and counting the tagged items 110 in the surgery tag list. The reader unit 200 comprises a reader communication unit 235, which is configured to communicate with a medical facility server 260 (e.g. via a network connection 265) and with one or more mobile controllers 240 (e.g. via a network connection 245). Network connections 245 and 265 may be wired network and/or wireless networks. In some non-limiting embodiments, the mobile controller 240 connects and communicates with the medical facility server 260 directly via network connection 248, which may include e.g. a wired network and/or a wireless network, cellular network, etc.

The reader unit 200 comprises a reader processor 207 configured to add and removes tagged items 110 from the surgery tag lists. The reader processor 207 calculates or determines a total count of the tagged items in the surgery tag list to analyze and determine whether a tagged item is missing. The reader processor 207 activates a locator antenna 220 to locate the missing tagged item. In some cases, the reader processor 207 activates the locator antenna 220 after receiving a command from the mobile controller 240 to activate the locator antenna 220.

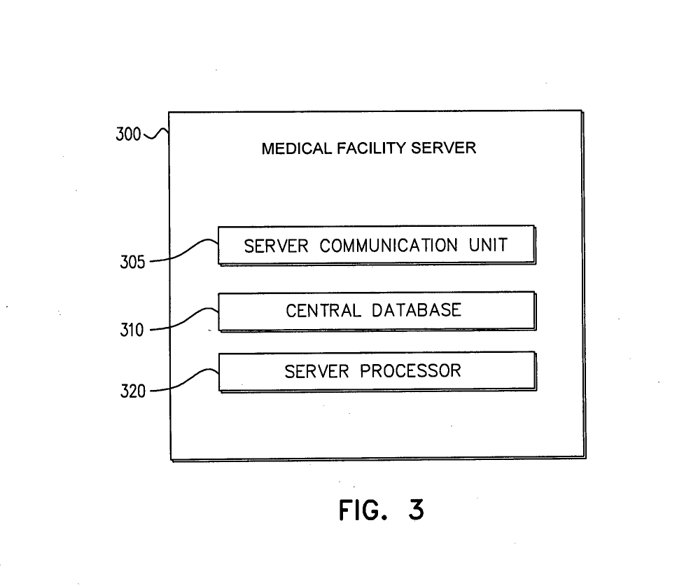

FIG. 3 shows a medical facility server, according to some exemplary embodiments of the subject matter. The medical facility server 300 comprises a server communication unit 305, which communicates with the one or more reader units 105 of FIG. 1 and the one or more mobile controllers 140 of FIG. 1. The server communication unit 305 receives updated surgery tag lists from the one or more reader units 105 and stores the surgery tag lists in a central database 310. Each time one of the surgery tag lists is updated by one of the reader units 105, it is transmitted to the medical facility server 300 and the updated surgery list is stored in the central database 310. For example, a surgery tag list may be updated when tagged items 110 of FIG. 1 are removed, and the updated surgery tag list is transmitted to the medical facility server 300 and stored in the central database 310.

The central database 310 stores a set of predetermined rules and conditions for activating alerts or notifications that are triggered when discrepancies are detected between tagged item total counts in the surgery tag lists received from the one or more reader units 105. The central database 310 stores the tagged item information.

The medical facility server 300 comprises a processor 320, which is configured to analyze received surgery tag lists to determine if a predetermined event is detected, or whether one or more rules or conditions of the set of rules and conditions are met in the analyzed surgery tag lists.

The predetermined set of rules may include, for example, a missing tagged item or an unaccounted tagged item is detected, too many items of a certain type are added to a surgery tag list (e.g., inserted into an operating room).

Examples of a predetermined event include: a total count higher than a first predetermined threshold, that indicates that a number of tagged items, or tagged items of a certain type that were inserted into the surgery tag list is higher than a predetermined quantity; a total count higher than a first predetermined threshold, that indicates that a number of tagged items removed from the surgery tag lists is higher than a predetermined quantity; a count of missing tags items is higher than a predetermined quantity, or the like

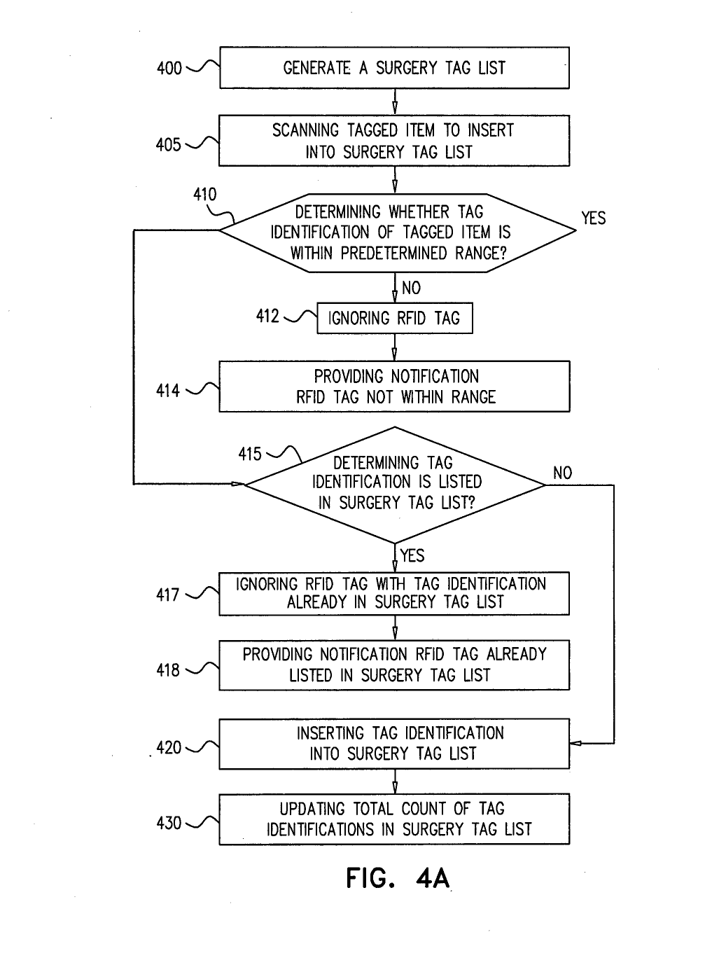

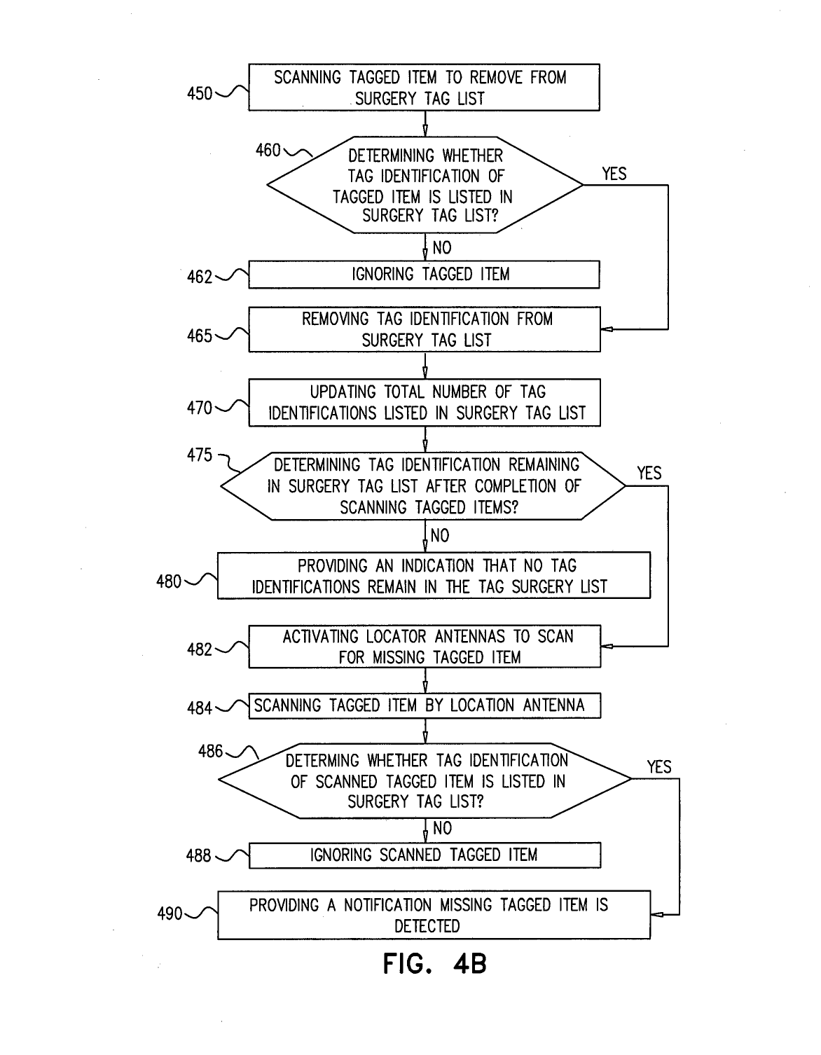

FIGS. 4A-4B are flow charts of methods for adding tagged items to and removing tagged items from a surgery tag list, according to some exemplary embodiments of the subject matter. FIG. 4A is a flow chart of a method for generating a surgery tag list and inserting a tagged item (e.g., associated with RFID tag information) into the surgery tag list, according to some exemplary embodiments of the subject matter.

Step 400 discloses generating a surgery tag list. The surgery tag list stores RFID tag data and/or information related to tagged items that are used during a medical procedure in an operating room 130 of FIG. 1. The surgery tag list is generated by a medical professional, e.g. a nurse, using, for example, the mobile controller 140 of FIG. 1.

Step 405 discloses scanning a tagged item 110 in order to insert it into the surgery tag list. The scanning is performed by a medical staff member, e.g. the nurse, using the handheld scanner 125 of FIG. 1. The reader unit 105 obtains the tag identification data from the handheld scanner 125 activated by a nurse in an operating room 130 of FIG. 1 and stores the tag identification data in the surgery tag list. The scanned data may be indicated or marked in the surgery tag list as items currently in use in the medical procedure taking place in the operating room.

Unique tag identification data may be assigned to each tagged item 110, a, for example during the manufacturing process. In some embodiments, the unique tag identification data may be a product serial number. The tag identification data is stored on the RFID tag 115 of FIG. 1, which is attached to the tagged item 110. The tagged item 110 is manufactured with other tagged items, for example 1,000 other tagged items. Each tagged item is provided with tag identification data within a predetermined identification range, which correlates to the tagged items manufactured together. For example, disposable sponges are provided a predetermined number range for recognizing the disposable sponges.

Step 410 discloses determining whether tag identification data of the tagged item 110 is within a predetermined range. When the tagged item 110 is scanned using the handheld scanner 125, the reader unit 105 receiving the inserted tag identification data may compare the obtained tag identification data to the predetermined identification range.

In step 412, if the scanned tag identification data is determined to be outside the predetermined identification range, the reader unit 105 ignores the obtained tag identification data.

Step 414 discloses providing a notification to a user (e.g., the medical staff) when the tag identification data is outside the predetermined identification range of values. Accordingly, the reader unit 105 provides a notification to the nurse scanning the tagged items, to inform the nurse that the tag identification data is not within the predetermined identification range. For example, the notification may be a visual and/or audible message stating “error, the scanned item was not identified.”

Step 415 discloses determining whether the tag identification data is listed as currently used in surgery tag list. The reader unit 105 compares the tag identification data of the scanned tagged item with the tag identification data of tagged items marked as currently used in the surgery tag list to ensure that the inserted tagged item associated with the tag identification data is not already listed in the surgery tag list).

Step 417 discloses ignoring an inserted RFID tag with tag identification data that is already in the surgery tag list. When the reader unit 105 determines that the tag identification data is already listed in the surgery tag list, the reader unit 105 ignores the tag identification data.

Step 418 discloses providing a notification the RFID tag is already listed in the surgery tag list. In some non-limiting cases, the reader unit 105 provides the notification is provided to inform the medical staff using the handheld scanner 125. For example the reader unit 105 transmits the notification to the mobile controller 140, which displays the notification to the medical staff. It should be noted that Steps 410-418 are optional and not necessary for performance of the method described herein.

Step 420 discloses inserting the tag identification data into the surgery tag list. The reader unit 105 inserts the tag notification data into the surgery tag list to store the tag notification data. In some embodiments, the tagged item associated with the inserted tag identification data may be indicated as presently in use in the medical procedure. For example, a timestamp and date may be stored in reader unit 105, indicating a time and date that the tagged item was scanned and added to the surgery tag list.

Step 430 discloses updating a total count of tagged items currently in use in the medical procedure, based on the tag identification data stored in the surgery tag list. The reader unit 105 updates the count of tagged items currently in use in the surgery tag list and sums the number of tagged items listed in the surgery tag list after the insertion of the tagged item 110 into the surgery tag list. The total count of tagged items currently in use in the medical procedure is continuously updated and maintained during the medical procedure.

FIG. 4B shows a method for performing a total count of tagged items currently present in the operating room during a medical procedure or after completing the medical procedure, according to some exemplary embodiments of the subject matter. Step 450 discloses scanning a tagged 110 item, in order to remove the tagged item 110 from surgery tag list. During the medical procedure or after the medical procedure, the tagged item 110 may be removed from the surgery tag list. For example, after the tagged item 110 is used during the medical procedure, it must be removed from the operating room 130 to prevent contamination. The tagged item 110 may be scanned, for example, by a medical professional using the handheld scanner 125, e.g. upon removal of the tagged item 110 from the operating room or after the tagged item 110 is removed from the operating room 130.

Step 460 discloses determining whether the tag identification data of the tagged item 110 is listed in the surgery tag list.

In Step 462, if the tag identification data of the tagged item 110 is not listed in the surgery tag list, the tagged item 110 is ignored.

Step 465 discloses removing the tag identification data from the surgery tag list, for example by erasing the related data record or marking it as removed or obsolete. If the tag identification data is listed in the surgery tag list, the reader unit 105 may remove the tagged item 110 from the surgery tag list by removing the tag identification data of the tagged item 110 from the surgery tag list. In some embodiments, the tag identification data may be marked or indicated as removed from the list of tagged items currently in use in the medical procedure. In some embodiments, a log maintaining the history of all tagged items that were inserted to a surgery tag list and removed from the surgery tag list may be stored, for example along with date/time data (e.g. a timestamp) of the insertion and removal from the surgery tag list.

Step 470 discloses updating the total count of the tag identification data listed in the surgery tag list. The reader unit 105 sums the number of tagged items listed in the surgery tag list after the removal of the tagged item 110 into the surgery tag list and updates the intermediate count of tagged items currently found in the surgery tag list.

In Step 475, the reader unit 105 may determine whether any tagged items are still in the surgery tag list. If no tag identification data remains in the tag surgery list (e.g. the list is empty, or all tagged items are marked as removed from the list), in Step 480 an indication is provided that the surgery tag list is empty. Upon or immediately after the completion of the medical procedure, the reader unit 105 validates that the tag surgery list is empty (e.g. the sum of the total count is zero) and provides an indication that all tagged items were removed from the surgery tag list.

Step 482 discloses automatically activating locator antennas 120 of FIG. 1 to scan for a missing tagged item. If the tag surgery list is not empty, e.g. a total count of the tag identification data in the surgery tag list is larger than zero, the reader unit 105 activates the locator antenna 120 in order to locate a missing tagged item.

Step 484 discloses scanning the tagged item 110 by the locator antenna 120. The locator antenna 120 scans for the missing tagged item, for example by emitting radio frequency waves to obtain the tag identification data from the RFID tag 115 of the missing tagged item. The locator antenna scans all RFID tags located within a scanning radius, for example, within a 5 meter radius of the locator antenna 120.

Step 486 discloses determining whether the tag identification data of a tagged item identified during the scanning of the locator antenna 120 is listed in the surgery tag list. When the locator antenna 120 scans the tagged item 110 within the scanning radius, it obtains the tag identification data of all scanned tagged items that are present in the scanning radius. The tag identification data is provided to the reader unit 105 to compare the tag identification data with the tag identification data listed in the surgery tag list.

If the tag identification data of the scanned tag item is not listed in the surgery tag list, in Step 488, the reader unit 105 ignores the scanned tagged item.

Step 490 discloses providing a notification that the missing tagged item was detected during the scan. If the tag identification data of the scanned tagged item matches the tag identification data of the missing tagged item listed in the surgery tag list, the reader unit 105 provides a notification that the missing tagged item has been detected. The notification may be provided to the mobile controller 140, which displays the notification (e.g. a visual and/or audible display) to the medical staff, e.g. a nurse.

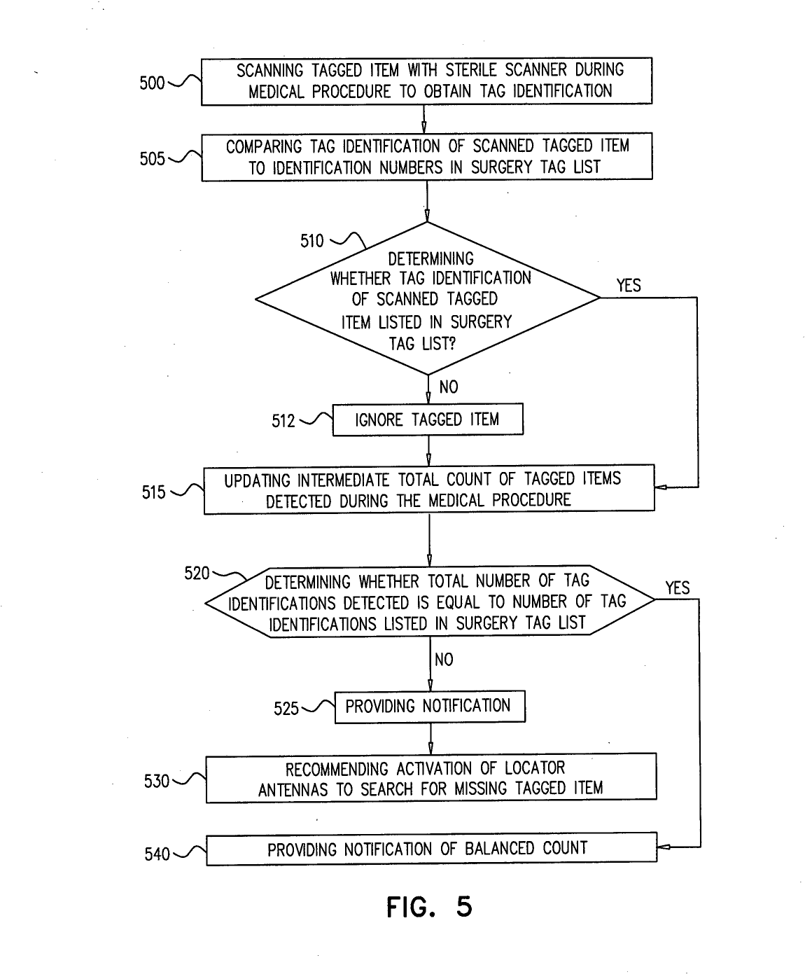

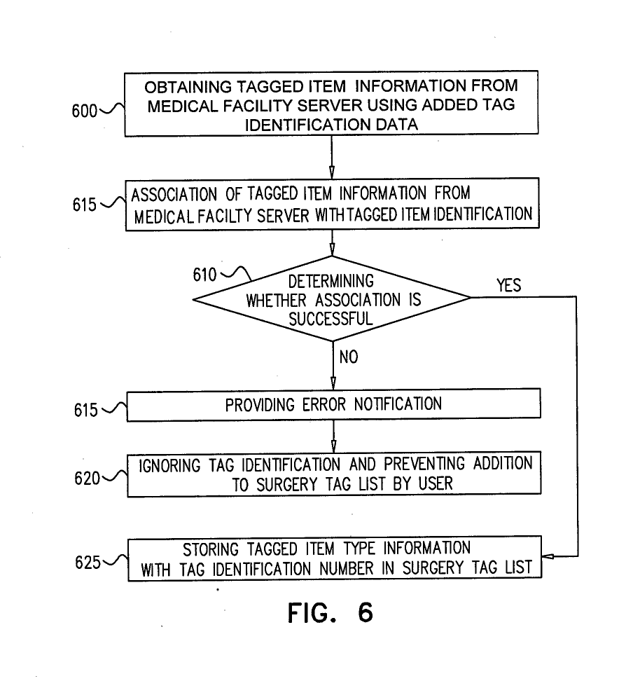

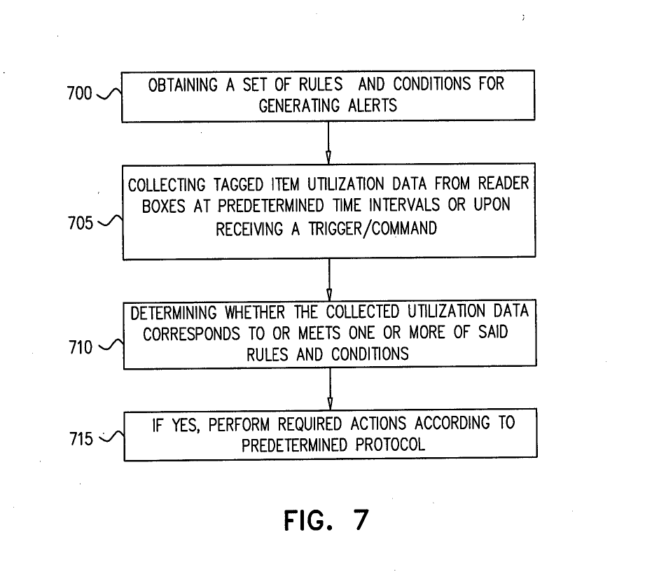

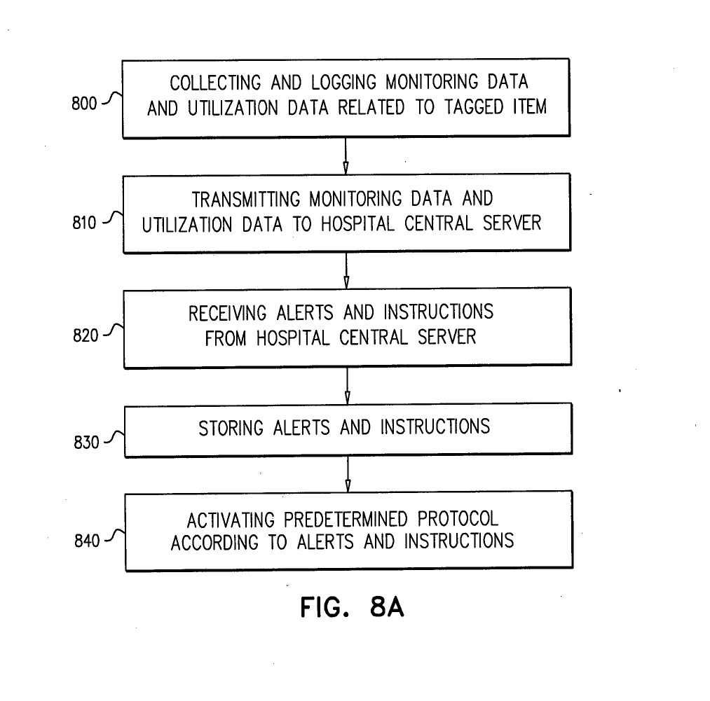

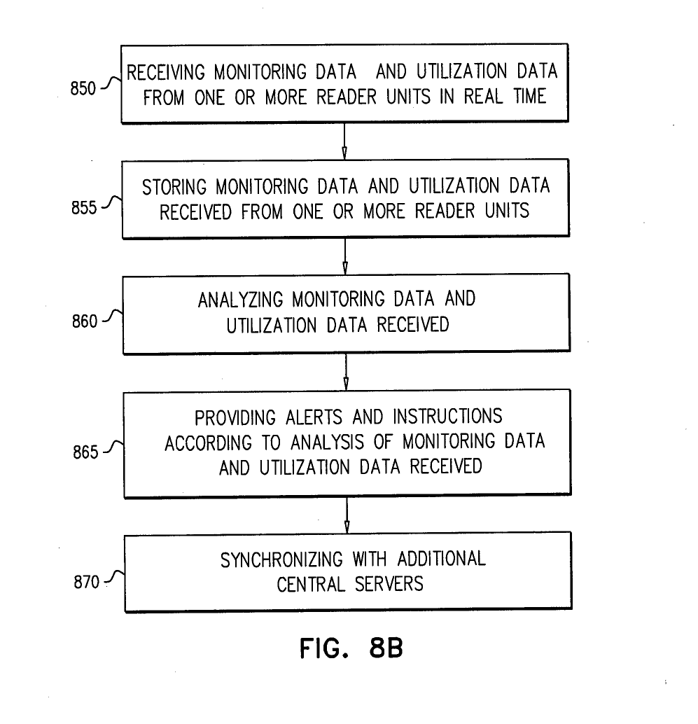

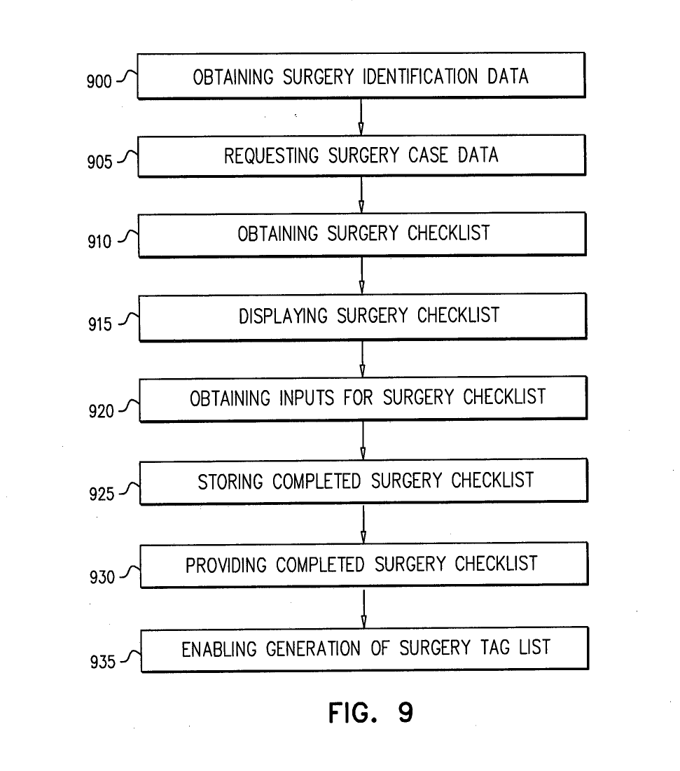

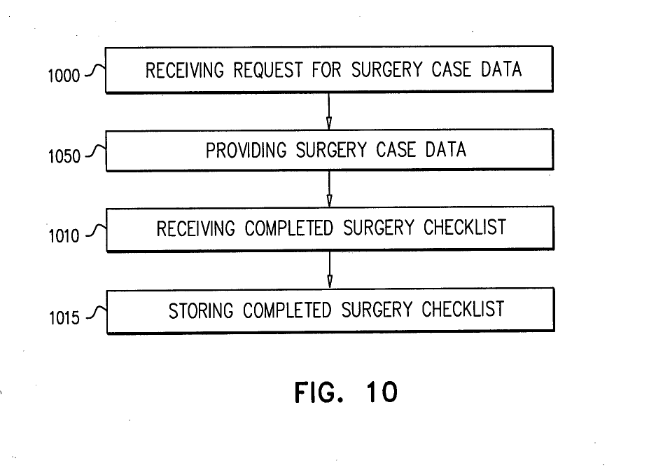

FIG. 5 discloses a method for performing an intermediate count of tagged items during a medical procedure, according to some exemplary embodiments of the subject matter. During the course of medical procedures, tagged items 110 of FIG. 1 are added to and removed from the operating room 130 of FIG. 1. To ensure that all tagged items 110 are accounted for in the operating room 130, an intermediate count is performed during the medical procedure, preferably by a scrub nurse.

Step 500 discloses scanning the tagged item 110 with handheld scanner 125 during a medical procedure to obtain the tag identification data. The scrub nurse uses the handheld scanner 125 covered with the sterile bag to scan the tagged items 110 in the operating room 130. The tag identification data is obtained by the reader unit 105 of FIG. 1 from the handheld scanner 125.

Step 505 discloses comparing the tag identification data of the scanned tagged item to the tag identification data listed in the surgery tag list. The reader unit 105 compares the tag identification data of the scanned tagged with tag identification data listed in the surgery tag list. For example, matching product serial numbers of the tagged item with listed product serial numbers stored in the surgery tag list.

Step 512 discloses ignoring the tagged item. If the tag identification data of the scanned tagged item does not match tag identification data listed in the surgery tag list the reader unit 105 ignores the tag identification data.

Step 515 discloses updating an intermediate total count of tagged items detected during the medical procedure.

Step 520 discloses determining whether the total count of tag identification data detected is equal to the count of tag identification data listed in the surgery tag list. The reader unit 105 analyzes and determines if the total count of tag identification data is equal to the count of tag identification data listed in the surgery tag list.

1. A system for monitoring tagged items in a medical facility, comprising:
one or more reader units configured to collect and store tracking data related to tagged items in real time, said monitoring data includes radio frequency tag identification data unique for each tagged item;
a medical facility server configured to receive the monitoring data from the one or more reader units, determine whether one or more predetermined rules or conditions are met, and trigger an action based on said determination;
wherein the monitoring data comprises identification data and utilization data related to tagged items, said tagged items intended for use in the medical facility.

2. The system of claim 1, wherein each reader unit of the one or more reader units comprises:
a radio frequency identification (RFID) input unit configured to obtain monitoring data stored in a RFID tag connected to a tagged item; said monitoring data comprises a tag identification data for identifying the tagged item;
a reader unit processor configured to continuously update a surgery tag list of tagged items in the operating room based on tag identification data; and
a reader unit database configured to store the monitoring data and utilization data of the tagged item.

3. The system of claim 1, wherein the medical facility server comprises:
a central database for storing the set of rules and conditions;
a central processor for analyzing collected monitoring data to determine whether an action should be triggered according to a predetermined protocol.

4. The system of claim 1, further comprising one or more locator antennas configured to penetrate human tissue and scan tagged items located within human tissue, wherein said one or more locator antennas are operationally connected to a reader unit of the one or more reader units.

5. The system of claim 1, further comprising one or more handheld scanners configured to scan tagged items in an operating room; said one or more handheld scanners are operationally connected to a reader unit of the one or more reader units.

6. The system of claim 5, wherein tagged items are scanned using a handheld scanner of the one or more handheld scanners to add a tagged item to the surgery tag list.

7. The system of claim 5, wherein tagged items are scanned by a handheld scanner of the one or more handheld scanners to remove a tagged item from the surgery tag list.

8. The system of claim 2, wherein the reader unit processor is configured to determine whether a tagged item is missing during a medical procedure or after completion thereof.

9. The system of claim 1, wherein the central processor is configured to determine whether a number of tagged items of a specific type which were used in a medical procedure is larger than a predetermined threshold.

10. The system of claim 1, further comprising one or more mobile controllers configured to communicate with the one or more reader units and with the medical facility server, said communication enabling the mobile controller to receive notifications from the medical facility server and from the reader unit, said notifications indicating that an action is required.

11. The system of claim 10, wherein the central processor is configured to trigger generation of a notification to one or more mobile controllers, based on said determination.

12. The system of claim 9, wherein the mobile controller is configured to initiate a command to the reader unit for activating a locator antenna configured to penetrate human tissue and scan RFID tags located within human tissue when a tagged item is determined as missing during a medical procedure.

13. Within a computerized system, a method for monitoring tagged items in a medical facility, the method comprising:
obtaining monitoring data from one or snore reader units located in one or more operating rooms,
determining, based on the monitoring data, whether one or more predetermined rules or conditions are met, and generating alerts based on said determination;
wherein the monitoring data comprises identification data and utilization data related to tagged items which are intended for use the medical facility.

14. The method of claim 13, comprising continuously updating the monitoring data during the medical procedure.

15. The method of claim 13, wherein the monitoring data comprises RFID tag identification data obtained by the one or more reader units by scanning the tagged item.

16. The method of claim 15, wherein the reader unit obtains the tag identification data from a handheld scanner.

17. The method of claim 13, wherein the reader unit obtains the tag identification data from a locator antenna configured to penetrate human tissue and scan RFID tags located within human tissue.

18. The method of claim 17, further comprising automatically activating the locator antenna when a tagged item is determined as missing.

19. The method of claim 13 further comprising associating tagged item information to tag identification data and storing the associated information.
